(19) United States
(12) Patent Application Publication
(10) Pub. No.: US 2003/0204247 A1
(43) Pub. Date: Oct. 30, 2003

(54) Semi-collapsed endoluminal graft membrane formed by polymer vapor deposition

(76) Inventors: Anthony Abbate, Novato, CA (US); Jacques Teisen, San Francisco, CA (US)

Correspondence Address: MEDTRONIC AVE, INC., 3576 UNOCAL PLACE, SANTA ROSA, CA, 95403, US

(21) Appl. No.: 10/133,037

(22) Filed: Apr. 25, 2002

(51) Int. Cl. A61F 2/06

(52) U.S. Cl. 623/1.29; 264/299; 427/237

(57) ABSTRACT

A polymer vapor deposition process is used to form a flexible thin-walled endoluminal graft membrane. The endoluminal graft membrane is easily radially compressed in an organized manner in combination with a stent. The endoluminal graft membrane is formed with a plurality of semi-collapsed legs and each semi-collapsed leg includes a plurality of preformed folds.

### BACKGROUND OF THE INVENTION

1. Field of the Invention

The present invention relates generally to stents and grafts, and more particularly to methods and structures for endoluminal grafts.

2. Description of Related Art

Various prior art methods have been used to make indwelling intravascular devices. See for example, U.S. Pat. No. 5,607,464 entitled “Intravascular Medical Device” of Schwartz, et al., issued on Mar. 4, 1997.

One problem associated with a combination of a stent and an endoluminal graft attached to the stent is collapsing the combination into an orderly small package that can be inserted into a blood vessel and successfully expanded. This limitation has limited the application of such combinations.

### SUMMARY OF THE INVENTION

According to one embodiment of the present invention, a polymer vapor deposition process is used to form a flexible thin-walled endoluminal graft membrane in a semi-collapsed state. One embodiment of an endoluminal graft membrane includes a plurality of legs. Each leg in the plurality of legs includes a plurality of preformed folds in a semi-collapsed state of the endoluminal graft membrane. The preformed folds facilitate an organized radial compression of the endoluminal graft membrane when the membrane is attached to a stent.

In one embodiment, at least one leg in the plurality of legs has a centerline. At least one preformed fold in the plurality of preformed folds of the at least one leg has a centerline oriented substantially perpendicular to the centerline of the at least one leg.

In another embodiment, at least one leg in the plurality of legs has a centerline. At least one preformed fold in the plurality of preformed folds of the at least one leg has a centerline that is an arc.

In still another embodiment, at least one leg in the plurality of legs has a centerline. At least one preformed fold in the plurality of preformed folds of the at least one leg is symmetric about the centerline.

In another embodiment, an endoluminal graft membrane includes a first plurality of legs with preformed folds in a semi-collapsed state of the endoluminal graft membrane, and a second plurality of legs without preformed folds in the semi-collapsed state of the endoluminal graft membrane. Each leg without preformed folds in the second plurality is positioned between a different pair of legs with preformed folds in the first plurality.

An endoluminal graft membrane polymer vapor deposition mold, in one embodiment according to the present invention, includes an endoluminal graft membrane pattern. The pattern includes a plurality of legs with preformed folds in a semi-collapsed state. In one embodiment, the plurality of legs comprises three legs and in another embodiment, four legs. The pattern, in another embodiment, also includes a second plurality of legs without preformed folds in the semi-collapsed state of the endoluminal graft membrane. Each leg without preformed folds in the second plurality is positioned between a different pair of legs with preformed folds.

In one embodiment of a method to make an endoluminal graft membrane in semi-collapsed state, a graft membrane pattern is fabricated in a mold. The graft membrane pattern includes at least a plurality of legs and each leg in said plurality of legs includes a plurality of preformed folds. The mold can be formed in longitudinal sections to facilitate release of the deposited endoluminal graft membrane.

In this embodiment of the method, the mold is placed in a polymer vapor deposition reaction chamber. As used herein, a polymer vapor deposition reaction chamber includes, but is not limited to, a chamber in any reactor capable of forming a polymer film having sufficient thickness to function as an endoluminal graft membrane including chemical vapor deposition and physical vapor deposition reactors. In the polymer vapor deposition reactor chamber, a polymer deposition process is used to form a thin-walled endoluminal graft membrane inside the endoluminal graft membrane pattern.

### BRIEF DESCRIPTION OF THE DRAWINGS

FIG. 1 is a top view of an endoluminal graft membrane with a plurality of semi-collapsed legs where each leg includes a plurality of preformed folds according to one embodiment of the present invention.

FIG. 2 is a perspective view one semi-collapsed leg of an endoluminal graft membrane with a plurality of preformed folds according to one embodiment of the present invention.

FIG. 3 is a top view of an endoluminal graft membrane with a first plurality of semi-collapsed legs where each leg includes a plurality of preformed folds and a second plurality of semi-collapsed legs without preformed folds, according to one embodiment of the present invention.

FIG. 4 is a top view of an endoluminal graft membrane with a first plurality of semi-collapsed legs where each leg includes a plurality of preformed folds and a second plurality of semi-collapsed legs without preformed folds, according to yet another embodiment of the present invention.

FIG. 5 is a partial illustration of a form including an endoluminal graft membrane pattern in a polymer vapor deposition reactor, according to one embodiment of the present invention.

FIG. 6 is a cross section view of an endoluminal graft membrane form with an endoluminal graft membrane pattern having a plurality of semi-collapsed legs where each leg includes a plurality of preformed folds according to one embodiment of the present invention.

FIG. 7 is a cross section view of an endoluminal graft membrane form with an endoluminal graft membrane pattern having a plurality of semi-collapsed legs where each leg includes a plurality of preformed folds according to another embodiment of the present invention.

FIG. 8 is a cross section view of an endoluminal graft membrane form with an endoluminal graft membrane pattern having a first plurality of semi-collapsed legs where each leg includes a plurality of preformed folds and a second plurality of semi-collapsed legs without preformed folds, according to one embodiment of the present invention.

FIG. 9 is a cross section view of an endoluminal graft membrane form with an endoluminal graft membrane pattern having a first plurality of semi-collapsed legs where each leg includes a plurality of preformed folds and a second plurality of semi-collapsed legs without preformed folds, according to another embodiment of the present invention.

FIG. 10 is a cross section view of an endoluminal graft membrane form with an endoluminal graft membrane pattern having a first plurality of semi-collapsed legs where each leg includes a plurality of preformed folds and a second plurality of semi-collapsed legs without preformed folds, according to yet another embodiment of the present invention.

In the Figures, elements with the same reference numeral are the same or equivalent elements. Also, the first digit of a reference numeral is the Figure number of the Figure in which the element with that reference numeral first appears.

### DETAILED DESCRIPTION

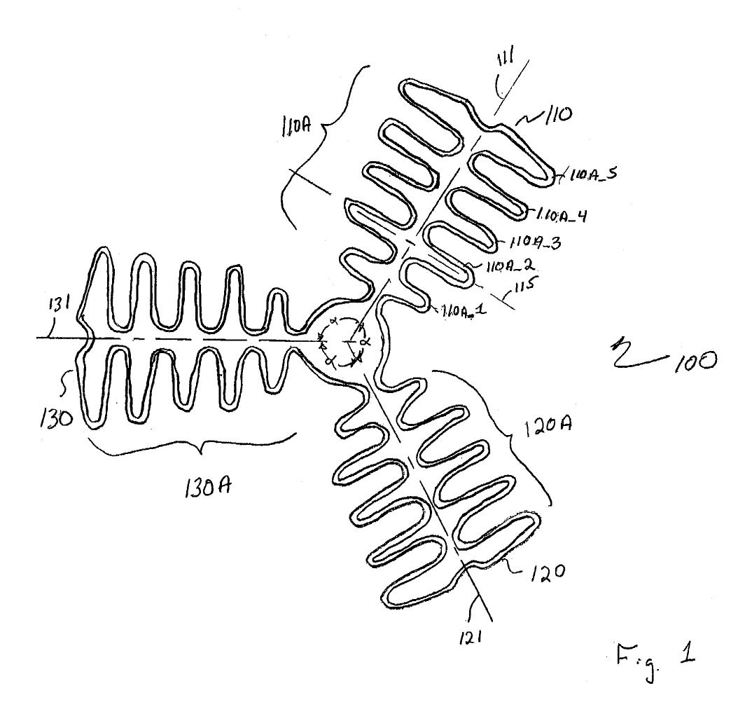

According to one embodiment of the present invention, a polymer vapor deposition process is used to form a flexible thin-walled endoluminal graft membrane 100 (FIG. 1) in a semi-collapsed state. As explained more completely below, endoluminal graft membrane 100 is easily radially compressed in an organized manner in combination with a stent.

Endoluminal graft membrane 100 is formed a plurality of legs, e.g., legs 110, 120, 130. Legs, as used herein, is synonymous with walls, wall sections, and other “leg” structures. In the semi-collapsed state, each leg 110, 120, 130 includes a plurality of preformed folds 110A, 120A 130A.

Pluralities of preformed folds 110A, 120A, 130A facilitate an organized radial collapse of endoluminal graft membrane 100 to a collapsed state. In some embodiments, the outside diameter of collapsed endoluminal graft membrane 100 is up to twenty times smaller than the outside diameter of expanded endoluminal graft membrane 100. This dramatic difference between the collapsed outside diameter and the expanded outside diameter makes it possible to use endoluminal graft membranes in applications where it previously was not possible because the prior art endoluminal graft membranes could not be collapsed into such a small size.

The number of legs, the number of preformed folds per leg, the shape of the preformed folds, and the orientation of the preformed folds with respect to the centerline of the leg in FIG. 1 are illustrative only, and are not intended to limit the configuration according to the invention to this specific embodiment. Various alternative embodiments are described below. In view of this disclosure, those of skill in the art can form an endoluminal graft membrane with a plurality of legs and a plurality of preformed folds per leg that are appropriate for a particular application.

FIG. 1 is a top view of one embodiment of endoluminal graft membrane 100 in a semi-collapsed state. Each centerline 111, 121, 131 of each leg 110, 120, 130 is oriented at an angle α with respect to the centerline of each of two adjacent legs, as illustrated in FIG. 1.

Each of semi-collapsed legs 110, 120, 130 has a similar shape and configuration. Accordingly, only one leg 110 of the three legs is considered in more detail, but this description is directly applicable to the other two legs 120 and 130.

Each of preformed folds 110A_1 to 110A_5 is symmetric about centerline 111 of leg 110. In addition, each of preformed folds 110A_1 to 110A_4, in a set of preformed folds in plurality of preformed folds 110A, is symmetric about a centerline of the preformed fold.

For example, preformed fold 110A_2 is symmetric about centerline 115 that is a straight line. Preformed fold centerline 115 is substantially perpendicular to leg centerline 111. It is stated that centerline 115 and centerline 111 are substantially perpendicular because the combination of manufacturing tolerances and the characteristics of the polymer material making up endoluminal graft membrane 100 may result in an angle that is not exactly perpendicular.

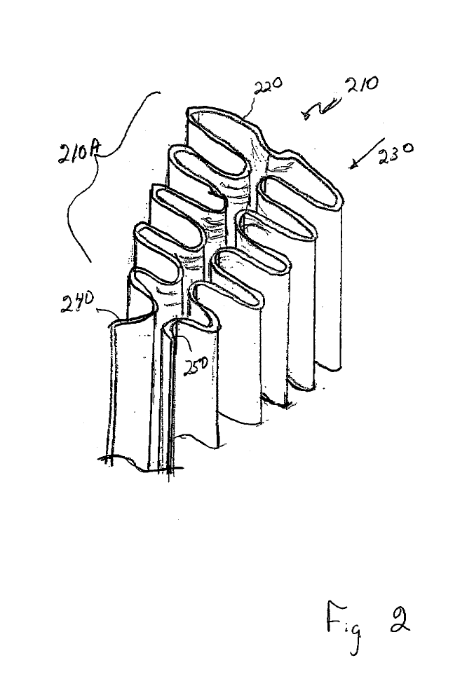

FIG. 2 is a perspective view of one semi-collapsed leg 210 with a plurality of preformed folds 210A of an endoluminal graft membrane, according to one embodiment of the invention. When a radial compression force 230 is applied to leg 210, the spaces between each of the preformed folds is compressed and leg 210 is compressed to reduce the outside diameter of the endoluminal graft membrane of which leg 210 is a part.

If outer folded edge surface 220 is affixed to an inside diameter of a stent using an adhesive, or alternatively a chemical reaction, when the stent is expanded, leg 210 unfolds and covers the inner diameter of the stent. The same compression and expansion of the endoluminal graft membrane are achieved if inner folded edge surfaces 240, 250 are attached to either the outside diameter of, or the inside diameter of a stent.

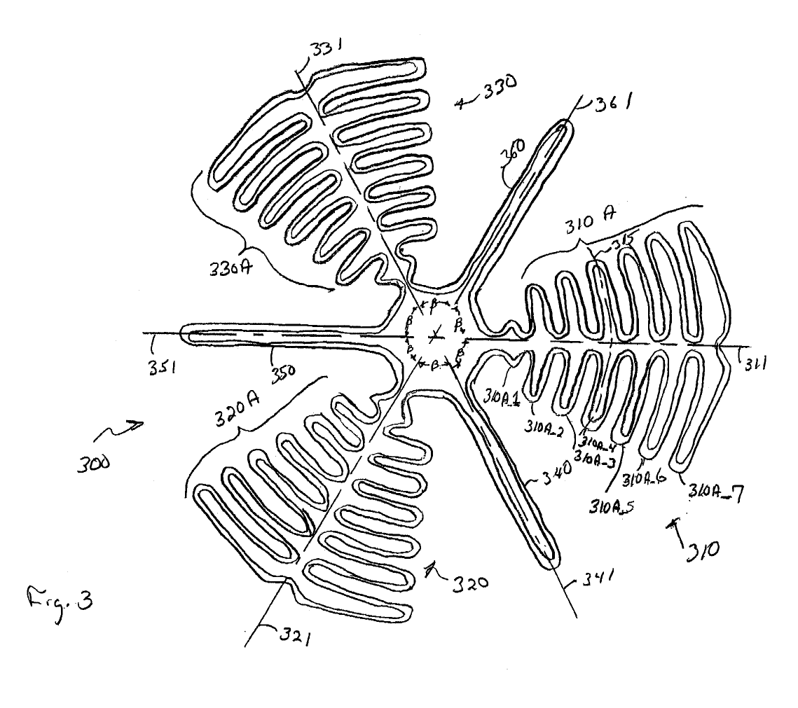

FIG. 3 is a top view of another embodiment of an endoluminal graft membrane 300 in a semi-collapsed state. Endoluminal graft membrane 300 is formed with a first plurality of semi-collapsed legs 310, 320, 330 with each leg having a plurality of preformed folds 310A, 320A, 330A. Endoluminal graft membrane 300 also includes a second plurality of radial legs 340, 350, 360 without preformed folds that are formed semi-collapsed. Each centerline 311, 321, 331, 341, 351, 361 of each leg 310, 320, 330, 340, 350, 360 is oriented at an angle β with respect to the centerline of each of two adjacent legs, as illustrated in FIG. 3.

Each of legs 310, 320, 330 in the first plurality of legs has a similar shape and configuration. Accordingly, only one leg 310 of the three legs is considered in more detail, but this description is directly applicable to the other two legs 320, 330.

Each of preformed folds 310A_1 to 310A_7 is symmetric about centerline 311 of leg 310. In this embodiment, a centerline of each of preformed folds 310A_1 to 310A_7 is an arc, i.e., centerline 315 of preformed fold 310A_4 is an arc.

Each of the three legs 340, 350, 360 in the second plurality of legs is positioned between a different pair of legs with preformed folds, i.e., legs 310 and 320, legs 320 and 330, and legs 330 and 310, respectively. The second plurality of legs 340, 350, 360 provide additional attachment points to the stent for endoluminal graft membrane 300 without compromising the organized compressibility of endoluminal graft membrane 300.

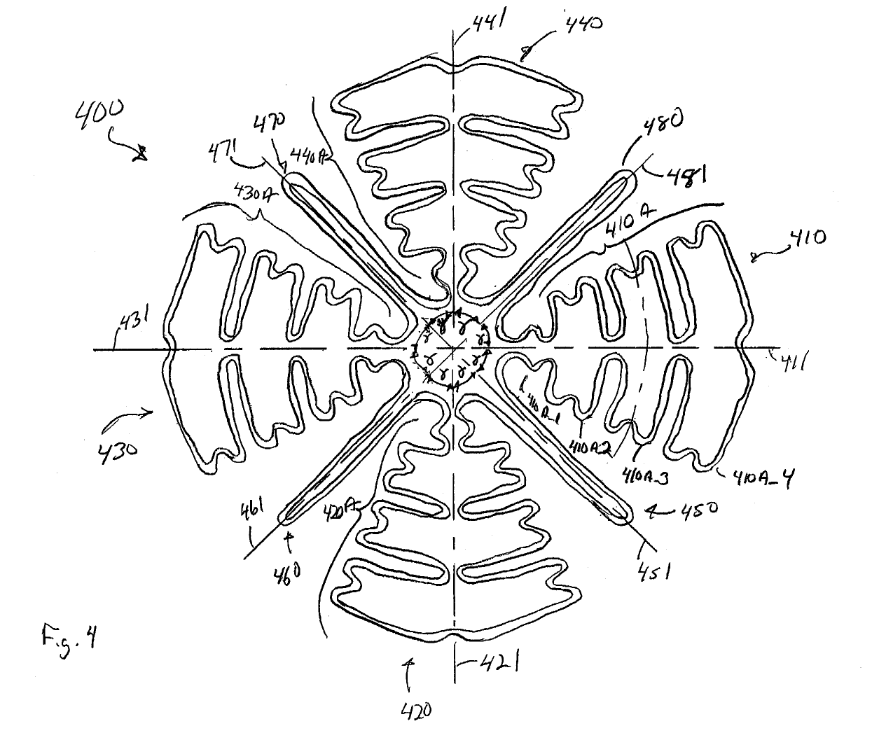

FIG. 4 is a top view of yet another embodiment of an endoluminal graft membrane 400 in a semi-collapsed state. Endoluminal graft membrane 400 is formed with a first plurality of semi-collapsed legs 410, 420, 430, 440 with each leg having a plurality of preformed folds 410A, 420A, 430A, 440A. Endoluminal graft membrane 400 also includes a second plurality of legs 450, 460, 470, 480 without preformed folds that are formed semi-collapsed. Each centerline 411, 421, 431, 441, 451, 461, 471, 481 of each leg 410, 420, 430, 440, 450, 460, 470, 480 is oriented at an angle y with respect to the centerline of each of two adjacent legs, as illustrated in FIG. 4.

Each of the four legs 410, 420, 430, 440 in the first plurality of legs has a similar shape and configuration. Accordingly, only one leg 410 of the four legs is considered in more detail, but this description is directly applicable to the other three legs 420, 430, 440. Each of preformed folds 410A_1 to 410A_4 is symmetric about centerline 411 of leg 410.

In this embodiment, effectively two folds in a leg of endoluminal graft membrane 300 are combined into a single fold. This removes some of the mass of endoluminal graft membrane 400 without compromising the structural integrity and so maintains the organized compressibility while providing an even greater number of attachment points to the stent.

Each of the four legs 450, 460, 470, 480 in the second plurality of legs is positioned between a different pair of legs with preformed folds, i.e., legs 410 and 420, legs 420 and 430, legs 430 and 440, and legs 440 and 410, respectively. The four legs 450, 460, 470, 480 of the second plurality of legs provide additional attachment points to the stent for endoluminal graft membrane 400 without compromising the organized compressibility of endoluminal graft membrane 400.

In one embodiment, a mold 510 is fabricated to form an endoluminal graft membrane pattern 500 for a graft membrane in a semi-collapsed state. In this embodiment, mold 510 is formed in a cylindrical piece of metal using electrical discharge machining (EDM). The metal can be tool steel, stainless steel, MP35N, or perhaps in another embodiment, a polymer or even PTFE. The process of forming such a pattern in a cylindrical metal piece is known to those of skill in the art. Mold 510 could be formed in longitudinal sections to facilitate release of the graft membrane that is formed.

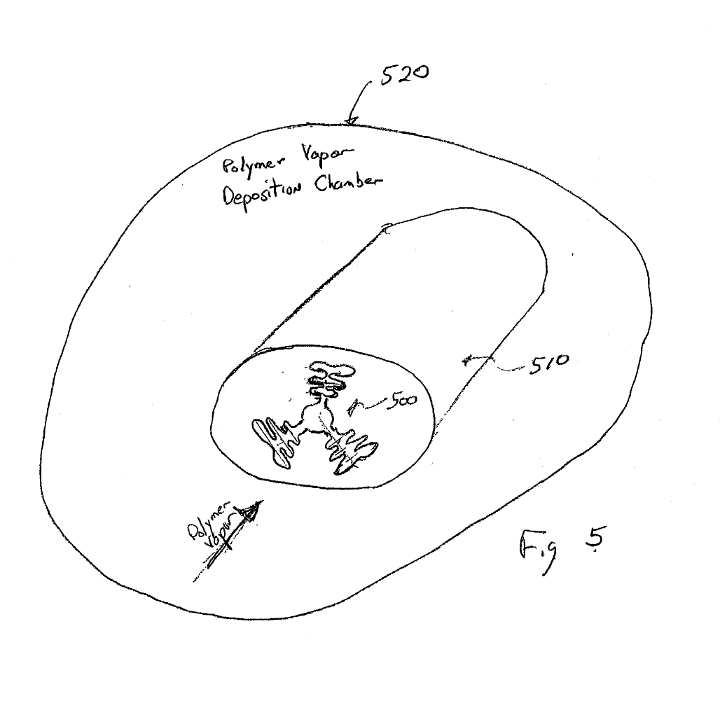

To form an endoluminal graft membrane, mold 510 (FIG. 5) is placed in a polymer vapor deposition reaction chamber 520. As used herein, a polymer vapor deposition reaction chamber includes, but is not limited to, a chamber in any reactor capable of forming a polymer film having sufficient thickness to function as an endoluminal graft membrane including chemical vapor deposition and physical vapor deposition reactors.

In polymer vapor deposition reaction chamber 520, a polymer deposition process is used to form a thin-walled endoluminal graft membrane inside endoluminal graft membrane pattern 500. Current polymer vapor deposition reactor technology permits forming an endoluminal graft membrane within mold 510 with wall dimensions less than 0.002 inches (0.005 cm) in uniform thickness. Polymer coatings that are suitable for use include polyurethane and PTFE. Both polymer vapor deposition reactors and the processes necessary to form thin-walled flexible polymer membranes within patterns of molds are known to those of skill in the art and so are a matter of empirical and iterative individual mold process development.

Hence, in one embodiment according to this invention, a method for creating an endoluminal graft membrane starts with forming an endoluminal graft membrane pattern in a mold. The endoluminal graft membrane pattern includes at least a plurality of legs. Each leg in the plurality of legs has a plurality of preformed folds in a semi-collapsed state of said endoluminal graft membrane.

The mold is placed in a polymer vapor deposition reaction chamber. A polymer vapor deposition process within the polymer vapor deposition reaction chamber is used to form a film within the mold having sufficient thickness to function as the endoluminal graft membrane.

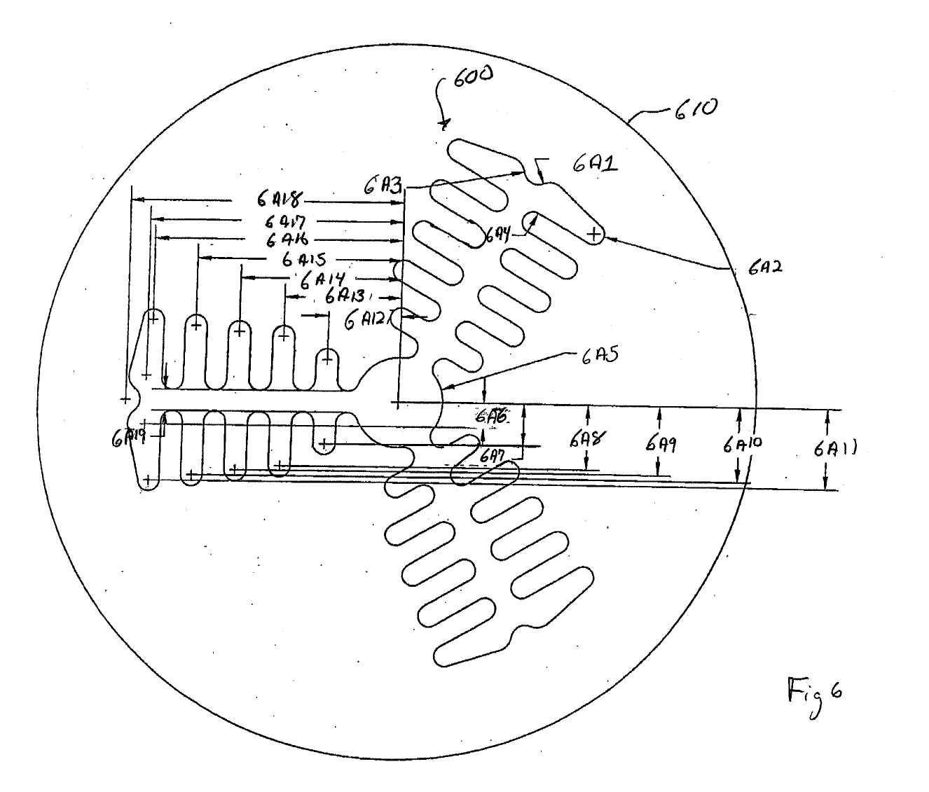

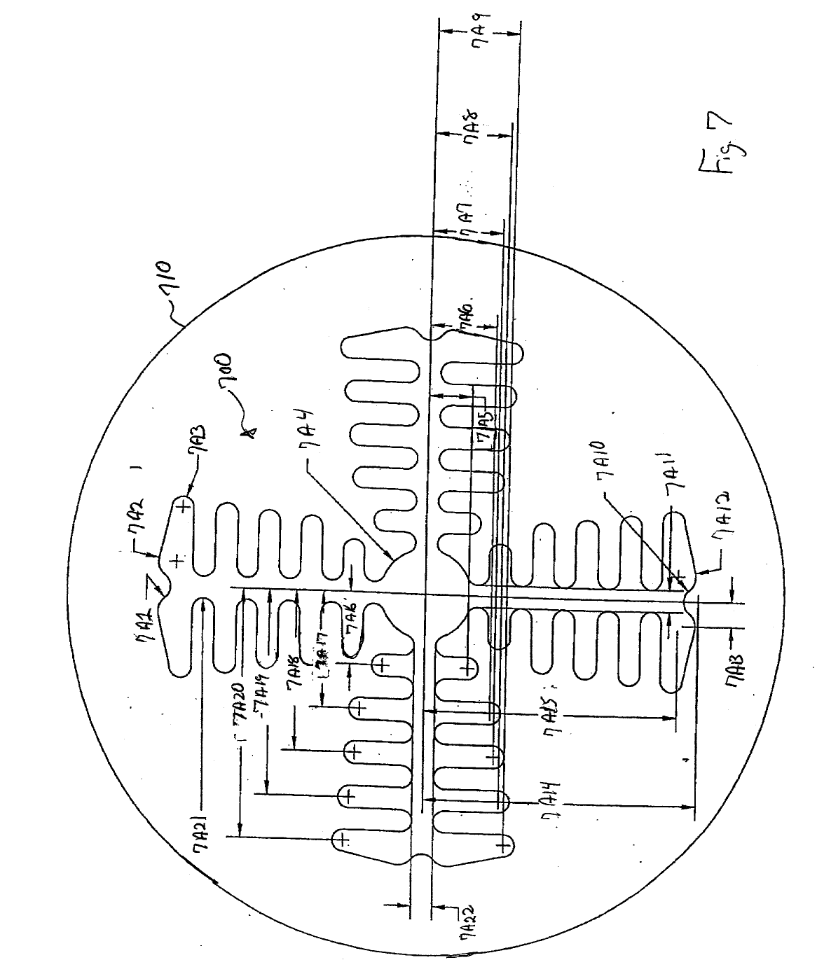

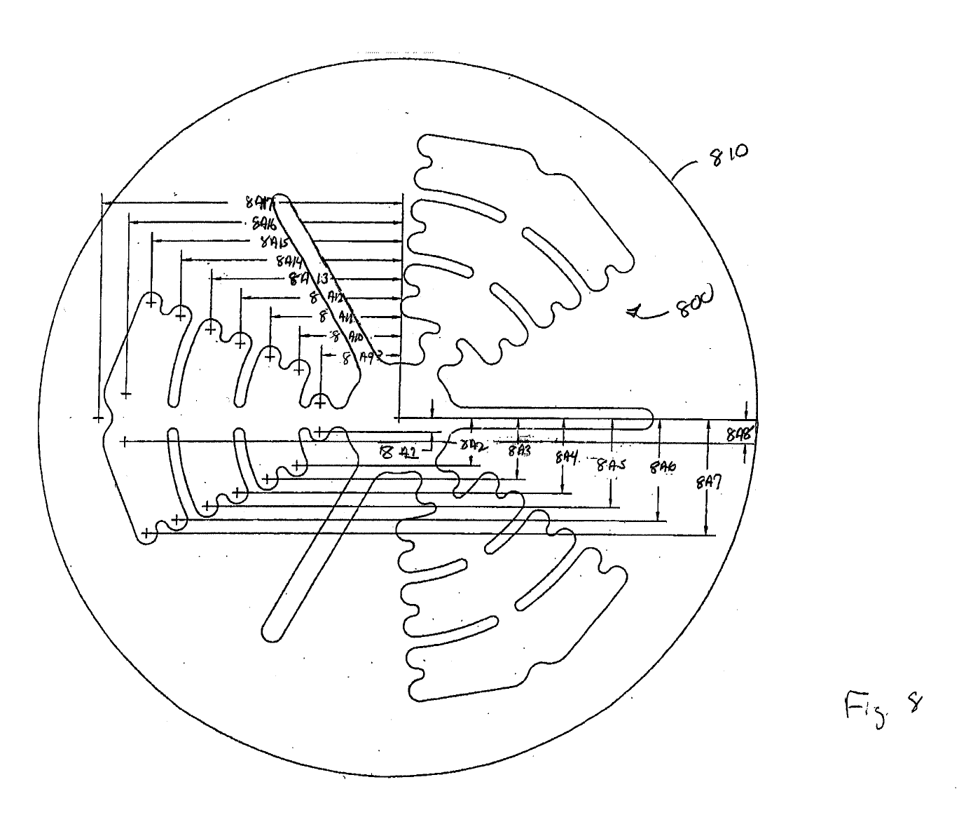

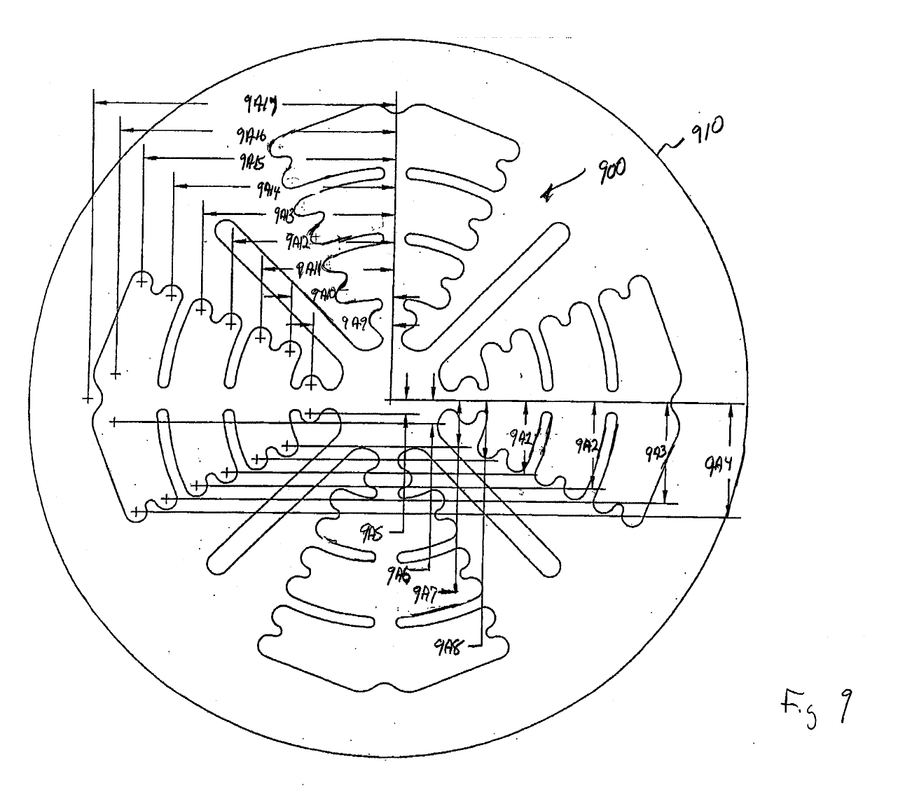

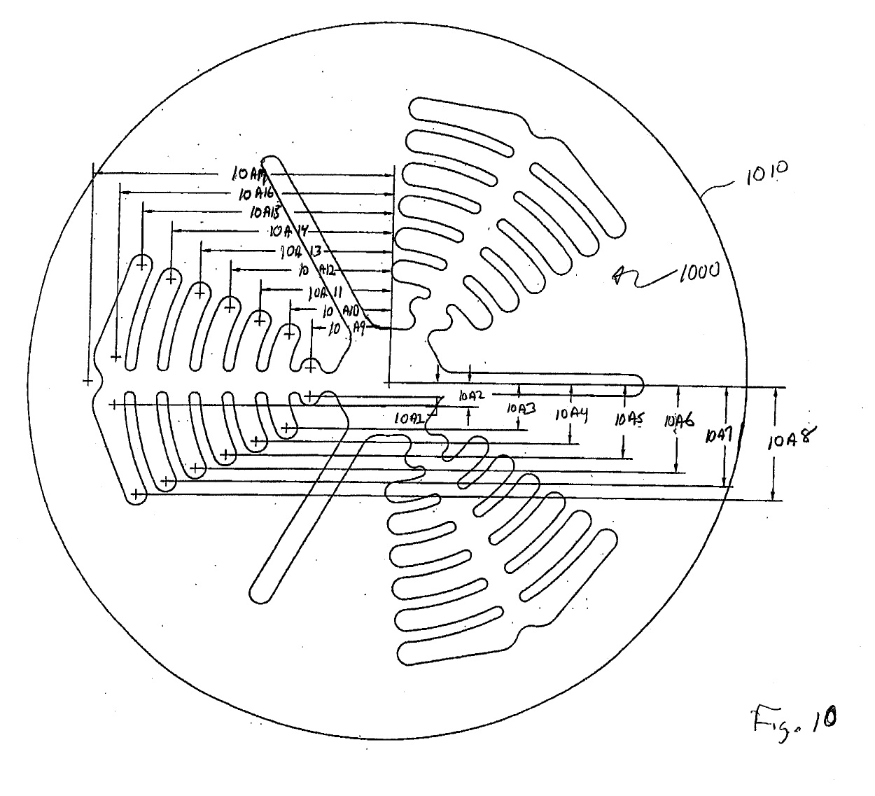

FIGS. 6 through 10 are cross-sectional drawings for various endoluminal graft membrane molds. Each of pictured molds 610, 710, 810, 910, 1010 is used to form an endoluminal graft membrane in a semi-collapsed state, as described above, using a polymer vapor deposition process. In view of the above description of the various endoluminal graft membranes, those of skill in the art understand the structure and characteristics of the endoluminal graft membrane formed using each of the different molds. Accordingly, the description is not repeated here.

Table 1 illustrates the dimensions for an embodiment of endoluminal graft membrane pattern 600 in mold 610. Pattern 600 is used to form membrane 100.

TABLE 1

| | |
|---|---|
| Reference | Dimension |

| Numeral | inches | cm |
|---|---|---|
| 6A1 | 0.0100(Radius) | 0.0254(Radius) |
| 6A2 | 0.0075(Radius) | 0.0191(Radius) |
| 6A3 | 0.0120(Radius) | 0.0305(Radius) |
| 6A4 | 0.0075(Radius) | 0.0191(Radius) |
| 6A5 | 0.0313(Radius) | 0.0795(Radius) |
| 6A6 | 0.0171 | 0.0434 |
| 6A7 | 0.0293 | 0.0744 |
| 6A8 | 0.0451 | 0.1146 |
| 6A9 | 0.0480 | 0.1219 |
| 6A10 | 0.0519 | 0.1318 |
| 6A11 | 0.0557 | 0.1415 |
| 6A12 | 0.0507 | 0.1288 |
| 6A13 | 0.0807 | 0.2050 |
| 6A14 | 0.1107 | 0.2812 |
| 6A15 | 0.1407 | 0.3574 |
| 6A16 | 0.1707 | 0.4336 |
| 6A17 | 0.1744 | 0.4430 |
| 6A18 | 0.1883 | 0.4783 |
| 6A19 | 0.0150 | 0.0381 |

Table 2 illustrates the dimensions for another embodiment of endoluminal graft membrane pattern 700 in mold 710.

TABLE 2

| | |
|---|---|
| Reference | Dimension |

| Numeral | inches | cm |
|---|---|---|
| 7A1 | 0.0100(Radius) | 0.0254(Radius) |
| 7A2 | 0.0120(Radius) | 0.0305(Radius) |
| 7A3 | 0.0075(Radius) | 0.0191(Radius) |
| 7A4 | 0.0625(Diameter) | 0.1588(Diameter) |
| 7A5 | 0.0293 | 0.0744 |
| 7A6 | 0.0451 | 0.1146 |
| 7A7 | 0.0480 | 0.1219 |
| 7A8 | 0.0519 | 0.1318 |
| 7A9 | 0.0557 | 0.1415 |
| 7A10 | 0.0201(Diameter) | 0.0511(Diameter) |
| 7A11 | 0.0150 | 0.0381 |
| 7A12 | 0.0240(Diameter) | 0.0610(Diameter) |
| 7A13 | 0.0171 | 0.0434 |
| 7A14 | 0.1883 | 0.4783 |
| 7A15 | 0.1744 | 0.4430 |
| 7A16 | 0.0507 | 0.1288 |
| 7A17 | 0.0807 | 0.2050 |
| 7A18 | 0.1107 | 0.2812 |
| 7A19 | 0.1407 | 0.3574 |
| 7A20 | 0.1707 | 0.4336 |
| 7A21 | 0.0075(Radius) | 0.0191(Radius) |
| 7A22 | 0.0150 | 0.0381 |

Table 3 illustrates the dimensions for yet another embodiment of endoluminal graft membrane pattern 800 in mold 810.

TABLE 3

| | |
|---|---|
| Reference | Dimension |

| Numeral | inches | cm |
|---|---|---|
| 8A1 | 0.0100 | 0.0254 |
| 8A2 | 0.0326 | 0.0828 |
| 8A3 | 0.0420 | 0.1067 |
| 8A4 | 0.0515 | 0.1308 |
| 8A5 | 0.0609 | 0.1547 |
| 8A6 | 0.0704 | 0.1788 |
| 8A7 | 0.0798 | 0.2027 |
| 8A8 | 0.0166 | 0.0422 |
| 8A9 | 0.0552 | 0.1402 |
| 8A10 | 0.0703 | 0.1786 |
| 8A11 | 0.0908 | 0.2306 |
| 8A12 | 0.1112 | 0.2824 |
| 8A13 | 0.1316 | 0.3343 |
| 8A14 | 0.1520 | 0.3861 |
| 8A15 | 0.1724 | 0.4379 |
| 8A16 | 0.1888 | 0.4796 |
| 8A17 | 0.2075 | 0.5271 |

Table 4 illustrates the dimensions for still yet another embodiment of endoluminal graft membrane pattern 900 in mold 910. Pattern 900 is used to form membrane 400

TABLE 4

| | |
|---|---|
| Reference | Dimension |

| Numeral | inches | cm |
|---|---|---|
| 9A1 | 0.0515 | 0.1308 |
| 9A2 | 0.0609 | 0.1547 |
| 9A3 | 0.0704 | 0.1788 |
| 9A4 | 0.0798 | 0.2027 |
| 9A5 | 0.0100 | 0.0254 |
| 9A6 | 0.0166 | 0.0422 |
| 9A7 | 0.0326 | 0.0828 |
| 9A8 | 0.0420 | 0.1067 |
| 9A9 | 0.0552 | 0.1402 |
| 9A10 | 0.0703 | 0.1786 |
| 9A11 | 0.0908 | 0.2306 |
| 9A12 | 0.1112 | 0.2824 |
| 9A13 | 0.1316 | 0.3343 |
| 9A14 | 0.1520 | 0.3861 |
| 9A15 | 0.1724 | 0.4379 |
| 9A16 | 0.1888 | 0.4796 |
| 9A17 | 0.2075 | 0.5271 |

Table 5 illustrates the dimensions for a still further embodiment of endoluminal graft membrane pattern 1000 in mold 1010. Pattern 1000 is used to form membrane 300.

TABLE 5

| | |
|---|---|
| Reference | Dimension |

| Numeral | inches | cm |
|---|---|---|
| 10A1 | 0.0100 | 0.0254 |
| 10A2 | 0.0166 | 0.0422 |
| 10A3 | 0.0326 | 0.0828 |
| 10A4 | 0.0420 | 0.1067 |
| 10A5 | 0.0515 | 0.1308 |
| 10A6 | 0.0609 | 0.1547 |
| 10A7 | 0.0704 | 0.1788 |
| 10A8 | 0.0798 | 0.2027 |
| 10A9 | 0.0552 | 0.1402 |
| 10A10 | 0.0703 | 0.1786 |
| 10A11 | 0.0908 | 0.2306 |
| 10A12 | 0.1112 | 0.2824 |
| 10A13 | 0.1316 | 0.3343 |
| 10A14 | 0.1520 | 0.3861 |
| 10A15 | 0.1724 | 0.4379 |
| 10A16 | 0.1888 | 0.4796 |
| 10A17 | 0.2075 | 0.5271 |

The various embodiments of the invention described herein are illustrative only. In view of this disclosure, those of skill in the art can make and use equivalent endoluminal graft membranes that utilize preformed folds to facilitate an organized radial compression of the graft membranes. While the molds are particularly suited for polymer vapor deposition, use of such molds with simple spraying technology with a tip spray arm that is guided through the molds described above may provide a less desirable, but still viable graft membrane.

We claim:

1. An endoluminal graft membrane comprising:
a plurality of legs wherein each leg in said plurality of legs comprises:
a plurality of preformed folds in a semi-collapsed state of said endoluminal graft membrane.

2. The endoluminal graft membrane of claim 1 wherein at least one leg in said plurality of legs has a centerline and at least one preformed fold in said plurality of preformed folds has a centerline oriented substantially perpendicular to said centerline of said at least one leg.

3. The endoluminal graft membrane of claim 1 wherein at least one leg in said plurality of legs has a centerline and at least one preformed fold in said plurality of preformed folds has a centerline that is an arc.

4. The endoluminal graft membrane of claim 1 wherein at least one leg in said plurality of legs has a centerline and at least one preformed fold in said plurality of preformed folds is symmetric about said centerline.

5. The endoluminal graft membrane of claim 1 further comprising:
at least one leg without preformed folds in said semi-collapsed state of said endoluminal graft membrane, and positioned between a pair of legs in said plurality of legs.

6. An endoluminal graft membrane comprising:
a first plurality of legs with preformed folds in a semi-collapsed state of said endoluminal graft membrane; and
a second plurality of legs without preformed folds in said semi-collapsed state of said endoluminal graft membrane wherein each leg without preformed folds in said second plurality is positioned between a different pair of legs with preformed folds in said first plurality.

7. The endoluminal graft membrane of claim 6 wherein at least one leg in said first plurality of legs has a centerline, and has at least one preformed fold having a centerline oriented substantially perpendicular to said centerline of said at least one leg.

8. The endoluminal graft membrane of claim 6 wherein at least one leg in said first plurality of legs has a centerline, and has at least one preformed fold, in said preformed folds of said at least one leg, has a centerline that is an arc.

9. The endoluminal graft membrane of claim 6 wherein at least one leg in said first plurality of legs has a centerline, and has at least one preformed fold, in said preformed folds of said at least one leg, that is symmetric about said centerline.

10. The endoluminal graft membrane of claim 6 wherein said second plurality of legs comprises three legs.

11. The endoluminal graft membrane of claim 6 wherein said second plurality of legs comprises four legs.

12. An endoluminal graft membrane polymer vapor deposition mold comprising:
an endoluminal graft membrane pattern comprising:
a plurality of legs with preformed folds in a semi-collapsed state.

13. The endoluminal graft membrane polymer vapor deposition mold of claim 12 wherein said plurality of legs comprises three legs.

14. An endoluminal graft membrane polymer vapor deposition mold of claim 12 wherein said plurality of legs comprises four legs.

15. The endoluminal graft membrane polymer vapor deposition mold of claim 12 wherein at least one leg in said plurality of legs has a centerline, and has at least one preformed fold having a centerline oriented substantially perpendicular to said centerline of said at least one leg.

16. The endoluminal graft membrane polymer vapor deposition mold of claim 12 wherein at least one leg in said plurality of legs has a centerline, and has at least one preformed fold having a centerline that is an arc.

17. The endoluminal graft membrane polymer vapor deposition mold of claim 12 wherein at least one leg in said first plurality of legs has a centerline, and has at least one preformed fold that is symmetric about said centerline.

18. The endoluminal graft membrane polymer vapor deposition mold of claim 12 further comprising:
a second plurality of legs without preformed folds in said semi-collapsed state of said endoluminal graft membrane wherein each leg without preformed folds in said second plurality is positioned between a different pair of legs with preformed folds.

19. A method of making an endoluminal graft membrane comprising:
placing a mold having a graft membrane pattern in a polymer vapor deposition reaction chamber wherein said graft membrane pattern includes at least a plurality of legs, wherein each leg in said plurality of legs comprises a plurality of preformed folds; and
depositing a polymer layer in said mold to form said endoluminal graft membrane wherein said endoluminal graft membrane includes said at least said plurality of legs.

20. The method of claim 19 further comprising:
forming said graft membrane pattern in said mold.
